(19) United States
(12) Patent Application Publication
(10) Pub. No.: US 2015/0374215 A1
(43) Pub. Date: Dec. 31, 2015

(54) DEVICE FOR EXAMINATION OF THE UPPER GASTROINTESTINAL TRACT

(71) Applicant: AMATEO Sp. z o.o., Kielce (PL)

(72) Inventor: Piotr Dulkiewicz, Lancut (PL)

(73) Assignee: AMATEO Sp. z o.o., Kielce (PL)

(21) Appl. No.: 14/434,004

(22) PCT Filed: Jul. 24, 2014
(86) PCT No.: PCT/PL2014/000085

§ 371 Date: Apr. 7, 2015

(30) Foreign Application Priority Data
Jun. 28, 2014 (PL) P.408688

(51) Int. Cl. A61B 1/04; A61B 1/00

(52) U.S. Cl. CPC A61B 1/041; A61B 1/00045

(57) ABSTRACT

The device for examination of the upper gastrointestinal tract equipped with a camera and connections system with the ability to connect a computer, video recorder, video printer and keyboard to describe the resulting image on the screen, characterized in that the device is in the form of a worm-like-shaped capsule equipped with the transparent dome with the camera (1) inside of the front part (2) of a shape similar to a part of a sphere passing smoothly into the flexural part (3) with possibility of extension and a rear part (4) with rounded edges.

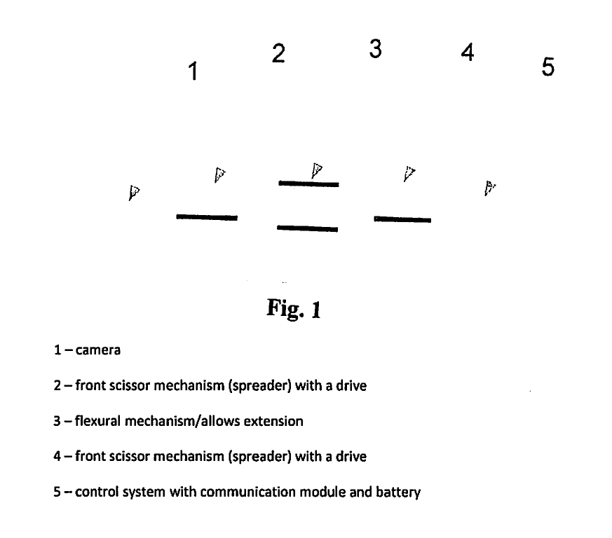

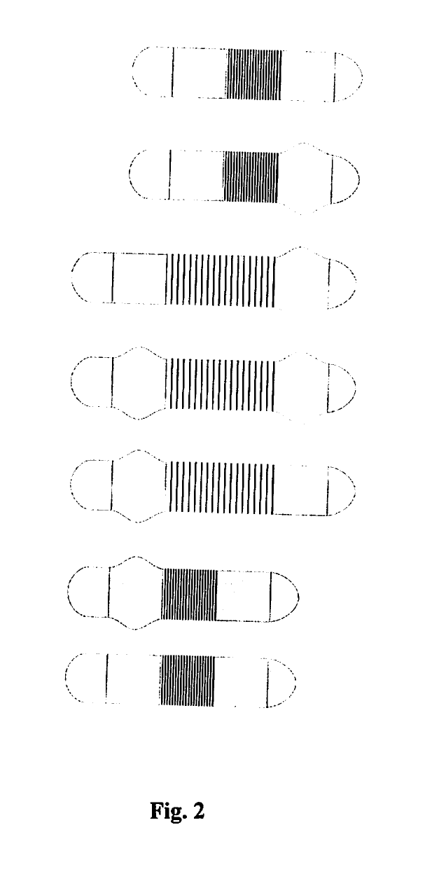

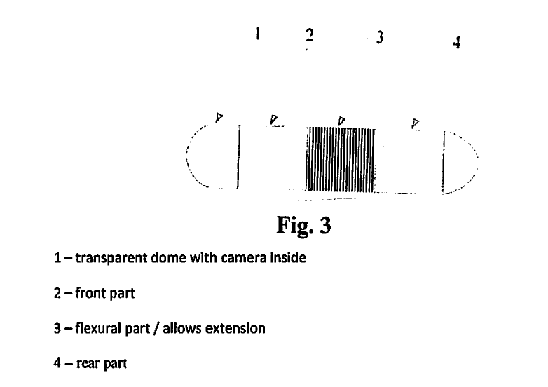

The present invention is a device for examination of the upper gastrointestinal tract dedicated for observations and measurements of the surfaces, which are difficult to access, used in medical diagnostics.

Fibre optic endoscope known from the Polish patent no. 165949 has an imaging system formed from a lens, an image transmitting unit in the form of a beam of optical fibres and an eyepiece. The imaging system is coupled to an illumination system formed of beam of optical fibres provided at an input with a grid projection optical system, which are located between this optical system and the light source lens. At an output of a coherent beam of optical fibres of the illumination system, the optical system is located on the grid projection for the observed object, at an acute angle relative to the optical axis of the imaging system. Eyepiece of an imaging system is coupled to a video-camera lens, the output of which is connected to the monitor and at the same time through an analog-digital converter to the input of the computer. In another version the imaging system is formed of a video-chip, which output is connected to the monitor and the analog-digital converter, which output is connected to the computer.

Another endoscope known from the Polish patent no. 165939 has also an imaging system, formed of a lens, a transmitting unit in the form of a beam of optical fibres and an eyepiece. The illumination system and additional illumination system are coupled to imaging system. Each of the illumination systems is formed of a non-coherent optical fibre beam equipped at the input with a lens and a light source. Additional illumination system has on the input of incoherent optical fibres beam an embedded grid made up of straight, parallel lines. The grid is located in front of the optical system impinging a grid on the test object at an acute angle relative to the direction of observation. Eyepiece is coupled to a video-camera, which output is connected via an analog-digital converter to the input of the computer. The tip of the endoscope can be equipped with the imaging system, in the form of a video-chip, which output is connected via an analog-digital converter to the input of the computer. Method of determining the surface condition of objects located in difficult to reach holes, cavities, recesses and a device for applying this method is known from the Polish patent description no. 166556. The method consists in that the test object is illuminated by the laser light via illumination system of known endoscope sequentially at two different angles relative to the axis of the direction of observation. The illuminated image of an object, for each of two consecutive illuminations, brought out through the imaging system, is projected on the photosensitive holographic plate forming a double-exposure hologram. Then, after induction and fixation, the resulting double-exposure hologram is played and using a video-camera converted to the analog signal, which after being converted to a digital signal is fed to the computer. The spatial dimensions of the object are determined in the computer based on the degree of overmodulation of black and white double-exposure hologram stripes via changes of object's dimensions in the axis of the direction of observation. The device has an imaging system in the form of a coherent beam of optical fibres, in the form of a lens system or Hopkins optics. The imaging system is coupled to an illumination system provided with a movable tip illuminating the test object at least two different angles relative to the axis of the direction of observation. The input of the illumination system is optically coupled by a light beam splitter to the output of the laser. The output of the imaging system is optically coupled with a light-sensitive, holographic plate located in the beam of the beam spreader optically coupled with the light beam splitter from a laser. The holographic plate is optically coupled to a video-camera, which output is connected via an analog-digital converter to the input of the computer.

Colored diagnostic system for mini-endoscope is known from U.S. Pat. No. 6,485,414, which is equipped with a black and white video system and a light source frontally mounted on the distal end of the endoscope, which contains at least two laser diodes, each having a different length of a wave illuminating the test object, wherein the light beam is directed along the axis of the endoscope. Light sources are operated by a driver sequentially incorporating the diodes illuminating the object, and the light reflected from the tested object is routed via optical fibres to the video processor, which receives succeeding black and white signals and generates a signal for each of the colours, and the display shows a colour image of the object.

The endoscope known from Japanese patent no. JP2002177197 has at the end of the endoscope in the body a frontally mounted light source in the form of an electroluminescent diode.

From the description of Polish utility model PL 67 076 Y1 3 an illumination system is known, which is provided with a light source comprising light-emitting diodes, mounted on a printed plate, which is placed in the body located at an end of the endoscope such that the light beam emitted by the light source is directed at an angle to the axis of the endoscope. In addition, the light source placed in the body is covered with a protective cover.

An endoscope, according to the utility model, can be widely used as an illuminator, in any type of speculums and surgical instruments having tool channels for sampling, called biopsy channels or others dedicated for introduction into the patient's body of micro-devices and micro-surgical devices, in particular, such as forceps or loops for minor surgeries.

The essence of the invention is characterized in that the remote-controlled machine in the form of a worm-like-shaped capsule is equipped with a transparent dome with a camera inside at the front end of a shape similar to a part of a sphere passing smoothly into the flexural part with a possibility of extension, and a rear part with rounded edges.

The advantage of a device is the possibility of crawling and movement within the upper gastrointestinal tract. The device is built in the form of an elongated capsule resembling a short worm -its body is moved by peristaltic movements, may also change the direction of crawl by bending the body. Introduced to the gastrointestinal tract can crawl both forwards and backwards providing video also in inversion especially in areas inaccessible to conventional endoscope (duodenum and esophagus). This additional information can be very important for diagnosing by visualizing i.e. the normally invisible changes in the lower sphincter of the stomach from the site of duodenum.

The present invention is shown in the embodiment on the schematic figure, on which was revealed the apparatus as seen from the side. The transparent dome with a camera 1 is placed in front part of device 2, behind which is a flexural part 3, construction of which allows extension of the capsule, the last part of the capsule is the rear part 4 of the gently contoured shape in the form of capsule.

Power supply of the device is a battery located inside the body.

Capsule front made of transparent plastic, the remainder part also of the material, but not necessarily transparent, but flexible, allowing for stretching (because the device is moving by the alternately expansion of front and rear part and an extension and bending of the central part).

The image is transmitted via radio waves to the external driver, which allows to remotely manage the device. Printing, saving an image is done by pressing a button on the handle of the device, it is also possible to connect a computer, video recorder, video printer and a keyboard to describe the resulting image on the screen. Furthermore, the device has a high resistance to damage caused by stomach acid environment due to the material used made of plastic.

### EXAMPLE

Capsule shaped device consists of four sections from plastic material coupled with one another and is equipped with power from the battery located inside the body. Image is directed to the camera, which is located in the transparent dome 1 on the input of the device.

The device features a high resolution of an image by using lenticular optics and a digital camera built in the dome of the device. The image of the device gives superior colour reproduction thanks to the well-known fully digital image processing by the video processor, it is an image without noise, sharp and clean, thanks to keeping the video signal in digital form. Printing, saving an image is done by pressing a button on the handle of the device, it is also possible to connect a computer, video recorder, video printer and a keyboard to describe the resulting image on the screen.

1. The device for examination of the upper gastrointestinal tract equipped with a camera and connections system with the ability to connect a computer, video recorder, video printer and keyboard to describe the resulting image on the screen, characterized in that the device is in the form of a worm-like-shaped capsule equipped with the transparent dome with the camera (1) inside of the front part (2) of a shape similar to a part of a sphere passing smoothly into the flexural part (3) with possibility of extension and a rear part (4) with rounded edges.

2. Device according to claim 1, characterized in that all the constituent elements of the body in the form of a capsule are made of plastic.
